(19) United States
(12) Patent Application Publication
(10) Pub. No.: US 2009/0036473 A1
(43) Pub. Date: Feb. 5, 2009

(54) NOVEL QUINAZOLINONE DERIVATIVES AND THEIR MEDICAL USE

(76) Inventors: William Dalby Brown, Søborg (DK); Carsten Jessen, Birkerod (DK); Joachim Demnitz, Kobenhavn (DK); Tino Dyhring, Solrod (DK); Dorte Strobaek, Parum (DK)

Correspondence Address: BIRCH STEWART KOLASCH & BIRCH, PO BOX 747, FALLS CHURCH, VA, 22040-0747, US

(21) Appl. No.: 12/278,091

(22) PCT Filed: Mar. 9, 2007
(86) PCT No.: PCT/EP2007/052239

§ 371 Date: Aug. 1, 2008

Related U.S. Application Data
(60) Provisional application No. 60/782,529, filed on Mar. 16, 2006.

(30) Foreign Application Priority Data
Mar. 15, 2006 (DK) PA 2006 00368

(51) Int. Cl. A61K 31/517; C07D 239/88

(52) U.S. Cl. 514/266.3; 544/287

(57) ABSTRACT

This invention relates to novel quinazolinone derivatives having medical utility, to use of the quinazolinone derivatives of the invention for the manufacture of a medicament, to pharmaceutical compositions comprising the quinazolinone derivatives of the invention, and to methods of treating a disorder, disease or a condition of a subject, which disorder, disease or condition is responsive to activation of Kᵥ7 channels.

### TECHNICAL FIELD

This invention relates to novel quinazolinone derivatives having medical utility, to use of the quinazolinone derivatives of the invention for the manufacture of a medicament, to pharmaceutical compositions comprising the quinazolinone derivatives of the invention, and to methods of treating a disorder, disease or a condition of a subject, which disorder, disease or condition is responsive to activation of Kᵥ7 channels.

### BACKGROUND ART

Potassium (K⁺) channels are structurally and functionally diverse families of K⁺-selective channel proteins, which are ubiquitous in cells, indicating their central importance in regulating a number of key cell functions. While widely distributed as a class, K⁺ channels are differentially distributed as individual members of this class or as families.

Recently a new family of potassium channels, the KCNQ channels, has attracted attention as target for therapeutic development. The human KCNQ1 channel was disclosed by Wang, Q et al. [Wang, Q et al. Nature Genet. 1996 12 17-23], the human KCNQ2 channel was disclosed by Biervert et al. [Biervert et al.; Science 1998 279 403-406]; the human KCNQ3 channel was disclosed by Schroeder et al. [Schroeder et at.; Nature 1998 396 687-690]; the human KCNQ4 channel was disclosed by Kubisch et al. [Kubisch et al.; Cell 1999 96 (3) 437-446]; and the human KCNQ5 channel was disclosed by Schroeder et al. [Schroeder et al.; J. Biol. Chem. 2000 275 (31) 24089-24095].

According to the latest nomenclature KCNQ1-KCNQ5 channels now are also designated Kᵥ7.1-Kᵥ7.5.

Due to the distribution of Kᵥ7 channels within the organism, Kᵥ7 channel modulators are considered potentially useful for the treatment or alleviation of conditions as diverse as epilepsy, anxiety, pain, migraine, tension type headache, CNS disorders, CNS damage caused by trauma, stroke or neurodegenerative illness or diseases, learning and cognitive disorders, motion and motor disorders, multiple sclerosis, heart failure, cardiomyopathia, cardiac disorders, inflammatory diseases, ophthalmic conditions, progressive hearing loss or tinnitus, obstructive or inflammatory airway diseases, for inducing or maintaining bladder control including the treatment or prevention of urinary incontinence.

WO 20041047738 describes arylcyclopropylcarboxylic amides useful as potassium channel (KCNQ) openers. However, the quinazolinone derivatives of the present invention are not suggested.

### SUMMARY OF THE INVENTION

It is an object of the present invention to provide novel quinazolinone derivatives having medical utility for combating disorders, diseases or conditions responsive to activation of Kᵥ7 channels.

In its first aspect the invention provides quinazolinone derivatives of Formula I

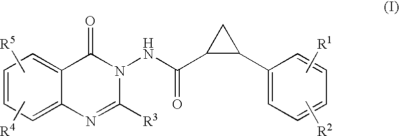

any of its isomers, or any mixture of its isomers, or a pharmaceutically-acceptable addition salt thereof, or an N-oxide thereof, wherein

R¹ and R², independently of each other, represent hydrogen, alkyl, cycloalkyl, halo, haloalkyl, hydroxy, alkoxy, haloalkoxy, amino, alkyl-carbonyl-amino, alkyl-sulfonyl, cyano or nitro;

R³ represents alkyl, cycloalkyl or alkoxy; and

R⁴ and R⁵, independently of each other represent hydrogen, alkyl, cycloalkyl, halo, haloalkyl, hydroxy, alkoxy, haloalkoxy, amino, alkyl-carbonyl-amino, nitro or cyano.

In another aspect the invention provides pharmaceutical compositions comprising a therapeutically effective amount of the quinazolinone derivative of the invention, or a pharmaceutically-acceptable addition salt thereof, or a prodrug thereof, together with one or more adjuvants, excipients, carriers and/or diluents.

Viewed from a third aspect the invention relates to the use of the quinazolinone derivative of the invention, or a pharmaceutically-acceptable addition salt thereof, for the manufacture of pharmaceutical compositions.

In a fourth aspects the invention provides a method of treatment, prevention or alleviation of a disease or a disorder or a condition of a living animal body, including a human, which disorder, disease or condition is responsive to activation of Kᵥ7 channels, which method comprises the step of administering to such a living animal body in need thereof, a therapeutically effective amount of the quinazolinone derivative of the invention, or a pharmaceutically-acceptable addition salt thereof.

Other objects of the invention will be apparent to the person skilled in the art from the following detailed description and examples.

### DETAILED DISCLOSURE OF THE INVENTION

The quinazolinone derivatives of the invention may be characterised by Formula I

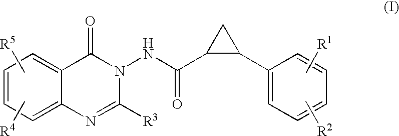

any of its isomers, or any mixture of its isomers, or a pharmaceutically-acceptable addition salt thereof, or an N-oxide thereof, wherein

R¹ and R², independently of each other, represent hydrogen, alkyl, cycloalkyl, halo, haloalkyl, hydroxy, alkoxy, haloalkoxy, amino, alkyl-carbonyl-amino, alkyl-sulfonyl, cyano or nitro;

R³ represents alkyl, cycloalkyl or alkoxy; and

R⁴ and R⁵, independently of each other, represent hydrogen, alkyl, cycloalkyl, halo, haloalkyl, hydroxy, alkoxy, haloalkoxy, amino, alkyl-carbonyl-amino, nitro or cyano.

In a preferred embodiment the quinazolinone derivative of the invention is a compound of Formula I, wherein R¹ and R², independently of each other, represent hydrogen, alkyl, cycloalkyl, halo, haloalkyl, hydroxy, alkoxy, haloalkoxy, amino, alkyl-carbonyl-amino, alkyl-sulfonyl, cyano or nitro.

In a more preferred embodiment R¹ and R², independently of each other, represent hydrogen, alkyl, in particular methyl, ethyl, propyl or isopropyl, cycloalkyl, halo, in particular fluoro, chloro, bromo or iodo, or trifluoromethyl.

In an even more preferred embodiment R¹ and R², independently of each other, represent hydrogen, alkyl, halo, in particular fluoro or chloro, or trifluoromethyl.

In a still more preferred embodiment R¹ and R², independently of each other, represent hydrogen, methyl, fluoro, chloro or trifluoromethyl.

In a yet more preferred embodiment R¹ represents hydrogen, alkyl, in particular methyl, cycloalkyl, halo, in particular fluoro or chloro, or haloalkyl, in particular trifluoromethyl; and R² represents hydrogen.

In a further more preferred embodiment R¹ represents hydrogen, alkyl, in particular methyl, halo, in particular fluoro or chloro, or trifluoromethyl; and R² represents hydrogen.

In a still further more preferred embodiment R¹ represents alkyl, in particular methyl; and R² represents hydrogen.

In a still further more preferred embodiment R¹ represents halo, in particular fluoro or chloro, or trifluoromethyl; and R² represents hydrogen.

In a still further more preferred embodiment R¹ represents fluoro or chloro; and R² represents hydrogen.

In a still further more preferred embodiment R¹ represents alkyl, in particular methyl, halo or trifluoromethyl; and R² represents halo, in particular fluoro or chloro.

In a still further more preferred embodiment R¹ represents methyl; and R² represents fluoro or chloro.

In a still further more preferred embodiment R¹ and R² both represent hydrogen.

In another preferred embodiment the quinazolinone derivative of the invention is a compound of Formula I, wherein R³ represents alkyl, cycloalkyl or alkoxy.

In a more preferred embodiment R³ represents alkyl, in particular methyl, ethyl, propyl or isopropyl.

In an even more preferred embodiment R³ represents isopropyl.

In a third preferred embodiment the quinazolinone derivative of the invention is a compound of Formula I, wherein R⁴ and R⁵, independently of each other, represent hydrogen, alkyl, cycloalkyl, halo, haloalkyl, hydroxy, alkoxy, haloalkoxy, amino, alkyl-carbonyl-amino, nitro or cyano.

In a more preferred embodiment R⁴ represents alkyl, cycloalkyl, halo, haloalkyl, hydroxy, alkoxy, haloalkoxy, amino, alkyl-carbonyl-amino, nitro or cyano; and R⁵ represent hydrogen.

In an even more preferred embodiment R⁴ and R⁵ both represent hydrogen.

In a still more preferred embodiment the quinazolinone derivative of the invention is

2-(4-Chloro-phenyl)-cyclopropanecarboxylic acid (2-isopropyl-4-oxo-4H-quinazolin-3-yl)-amide;

2-(4-Fluoro-phenyl)-cyclopropanecarboxylic acid (2-isopropyl-4-oxo-4H-quinazolin-3-yl)-amide;

2-(3-Fluoro-4-methyl-phenyl)-cyclopropanecarboxylic acid (2-isopropyl-4-oxo-4H-quinazolin-3-yl)-amide;

2-Phenyl-cyclopropanecarboxylic acid (2-isopropyl-4-oxo-4H-quinazolin-3-yl)-amide;

2-(4-Chloro-phenyl)-cyclopropanecarboxylic acid (2-isopropyl-4-oxo-4H-quinazolin-3-yl)-amide;

2-(4-Fluoro-phenyl)-cyclopropanecarboxylic acid (2-isopropyl-4-oxo-4H-quinazolin-3-yl)-amide;

2-p-Tolyl-cyclopropanecarboxylic acid (2-isopropyl-4-oxo-4H-quinazolin-3-yl)-amide;

2-(3-Fluoro-phenyl)-cyclopropanecarboxylic acid (2-isopropyl-4-oxo-4H-quinazolin-3-yl)-amide; or 2-p-Tolyl-cyclopropanecarboxylic acid (2-isopropyl-4-oxo-4H-quinazolin-3-yl)-amide;

or a pharmaceutically-acceptable addition salt thereof.

In a yet more preferred embodiment the quinazolinone derivative of the invention is

(Cis)-2-(4-Chloro-phenyl)-cyclopropanecarboxylic acid (2-isopropyl-4-oxo-4H-quinazolin-3-yl)-amide;

(Trans)-2-(4-Fluoro-phenyl)-cyclopropanecarboxylic acid (2-isopropyl-4-oxo-4H-quinazolin-3-yl)-amide;

(Trans)-2-(3-Fluoro-4-methyl-phenyl)-cyclopropanecarboxylic acid (2-is-propyl-4-oxo-4H-quinazolin-3-yl)-amide;

(Trans)-2-Phenyl-cyclopropanecarboxylic acid (2-isopropyl-4-oxo-4H-quinazolin-3-yl)-amide;

(Trans)-2-(4-Chloro-phenyl)-cyclopropanecarboxylic acid (2-isopropyl-4-oxo-4H-quinazolin-3-yl)-amide;

(Cis)-2-(4-Fluoro-phenyl)-cyclopropanecarboxylic acid (2-isopropyl-4-oxo-4H-quinazolin-3-yl)-amide;

(Cis)-2-p-Tolyl-cyclopropanecarboxylic acid (2-isopropyl-4-oxo-4H-quinazolin-3-yl)-amide;

(Cis)-2-(3-Fluoro-phenyl)-cyclopropanecarboxylic acid (2-isopropyl-4-oxo-4H-quinazolin-3-yl)-amide; or

(Trans)-2-p-Tolyl-cyclopropanecarboxylic acid (2-isopropyl-4-oxo-4H-quinazolin-3-yl)-amide;

or a pharmaceutically-acceptable addition salt thereof.

In a most preferred embodiment the quinazolinone derivative of the invention is

(Cis)-2-(4-Chloro-phenyl)-cyclopropanecarboxylic acid (2-isopropyl-4-oxo-4H-quinazolin-3-yl)-amide;

(Cis)-2-(4-Fluoro-phenyl)-cyclopropanecarboxylic acid (2-isopropyl-4-oxo-4H-quinazolin-3-yl)-amide;

Cis)-2-p-Tolyl-cyclopropanecarboxylic acid (2-isopropyl-4-oxo-4H-quinazolin-3-yl)-amide; or

(Cis)-2-(3-Fluoro-phenyl)-cyclopropanecarboxylic acid (2-isopropyl-4-oxo-4H-quinazolin-3-yl)-amide;

or a pharmaceutically-acceptable addition salt thereof.

Any combination of two or more of the embodiments described herein is considered within the scope of the present invention.

### Definition of Substituents

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In another preferred embodiment of this invention alkyl represents a C₁₋₃-alkyl group, which may in particular be methyl, ethyl, propyl or isopropyl.

In the context of this invention a cycloalkyl group designates a cyclic alkyl group, preferably containing of from three to seven carbon atoms (C₃₋₇-cycloalkyl), including cyclopropyl, cyclobutyl, cyclopentyl, cyclohexyl and cycloheptyl.

In the context of this invention halo represents fluoro, chloro, bromo or iodo. Thus a trihalomethyl group represents e.g. a trifluoromethyl group, a trichloromethyl group, and similar trihalo-substituted methyl groups.

In the context of this invention a haloalkyl group designates an alkyl group as defined herein, which alkyl group is substituted one or more times with halo. Preferred haloalkyl groups of the invention include trihalomethyl, preferably trifluoromethyl.

In the context of this invention a hydroxy-alkyl group designates an alkyl group as defined above, which hydroxy-alkyl group is substituted with one or more hydroxy groups. Examples of preferred hydroxy-alkyl groups of the invention include 2-hydroxy-ethyl, 3-hydroxy-propyl, 4-hydroxy-butyl, 5-hydroxy-pentyl and 6-hydroxy-hexyl.

In the context of this invention an alkoxy group designates an “alkyl-O—” group, wherein alkyl is as defined above. Examples of preferred alkoxy groups of the invention include methoxy and ethoxy.

In the context of this invention an alkyl-carbonyl-amino group designates an “alkyl-CO—NH—” group, wherein alkyl is as defined above. Preferred alkyl-carbonyl-amino groups of the invention include acetamido.

### Pharmaceutically Acceptable Salts

The quinazolinone derivatives of the invention may be provided in any form suitable for the intended administration. Suitable forms include pharmaceutically (i.e. physiologically) acceptable salts, and pre- or prodrug forms of the quinazolinone derivatives of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

Additional examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzene-sulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

### Isomers

It will be appreciated by those skilled in the art that the quinazolinone derivatives of the present invention may exist in different stereoisomeric forms, including enantiomers, diastereomers, as well as geometric isomers (cis and trans isomers). The invention includes all such isomers and any mixtures thereof including racemic mixtures.

Preferred isomers of the invention are the cis isomers.

Racemic forms can be resolved into the optical antipodes by known methods and techniques. As the compounds of the invention include chiral carboxylic acids as intermediate compounds, one way of separating the enantiomeric acids is by use of an optically active amine, and liberating the diastereomeric resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphor-sulphonate) salts for example.

The quinazolinone derivatives of the present invention may also be resolved by the formation of diastereomeric amides by reaction of the chemical compounds of the present invention with an optically active activated amine such as that derived from (+) or (−)α-methylbenzylamine or the like.

Additional methods for the resolving the optical isomers are known in the art. Such methods include those described by Jaques J. Collet A, & Wilen S in “Enantiomers, Racemates, and Resolutions”, John Wiley and Sons, New York (1981).

Optical active compounds can also be prepared from optical active starting materials.

### Methods of Preparation

The quinazolinone derivatives of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The quinazolinone derivatives of the invention have been found useful as modulators of the Kᵥ7 (KCNQ) potassium channels. At present five such channels are known, i.e. the Kᵥ7.1 (KCNQ1) channel, the Kᵥ7.2 (KCNQ2) channel, the Kᵥ7.3 (KCNQ3) channel, the Kᵥ7.4 (KCNQ4) channel, and the Kᵥ7.5 (KCNQ5) channel, and heteromeric combinations hereof. Moreover, the modulatory activity may be inhibitory (i.e. inhibitory activity) or stimulatory (i.e. activating activity).

The modulatory activity may be determined using conventional methods, e.g. binding or activity studies, known in the art, or as described in the working examples.

In a preferred embodiment the quinazolinone derivatives of the invention show stimulating activity at Kᵥ7.2, Kᵥ7.3, Kᵥ7.4 and/or Kᵥ7.5 potassium channels, and heteromeric combinations hereof. Preferred compounds of the invention are selective, preferably showing Kᵥ7.2, Kᵥ7.2+Kᵥ7.3, and/or Kᵥ7.4 potassium channel activation.

Accordingly, the compounds of the invention are considered useful for treatment, prevention or alleviation of a disease or a disorder or a condition of a living animal body, including a human, which disorder, disease or condition is responsive to modulation of a Kᵥ7 potassium channel.

Due to the distribution of KCNQ channels within the organism, KCNQ channel modulators are considered useful for the treatment or alleviation of conditions as diverse as an affective disorder, neuro-physiological disorder, anxiety, depression, a bipolar disorder, mania, a sleep disorder, addiction, an eating disorder, a phobia, Parkinson's disease, a mood disorder, a psychotic disorder, a compulsive behaviour, mania, psychosis, schizophrenia, dementia, Alzheimer's disease, epilepsy, convulsions, seizures, seizure disorders, tremor, muscle spasms, myasthenia gravis, a motor neuron disease, motion and motor disorders, a Parkinson-like motor disorder, multiple sclerosis, amyelotrophic lateral sclerosis (ALS), HIV dementia, Huntington's disease, Pick's disease, torsades de pointes, functional bowel disorders, neurodegenerative disorders, CNS damage caused by trauma, stroke or neurodegenerative illness or diseases, ataxia, myokymia, spasticity, learning and cognitive disorders, memory dysfunction, memory impairment, age-associated memory loss, Down's syndrome, pain, acute pain, chronic (persistant) pain, mild pain, moderate or severe pain, neuropathic pain, central pain, pain related to diabetic neuropathy, to postherpetic neuralgia, to peripheral nerve injury or to drug addiction, somatic pain, visceral pain or cutaneous pain, pain caused by inflammation or by infection, postoperative pain, phantom limb pain, chronic headache, migraine, migraine-related disorders, tension-type headache, heart failure, cardiac disorders, cardiomyopathia, cardiac arrhythmia, cardiac ischaemia, long QT syndrome, inflammatory diseases or conditions, inflammatory bowel disease, Crohn's disease, ulcerative colitis, Creutzfeld-Jacobs disease, an obstructive or inflammatory airway disease, asthma, an airway hyper reactivity, pneumoconiosis, aluminosis, anthracosis, asbestosis, chalicosis, ptilosis, siderosis, silicosis, tabacosis, byssinosis, chronic obstructive pulmonary disease (COPD), excerbation of airways hyper reactivity, cystic fibrosis, progressive hearing loss, tinnitus, a drug-dependence or drug-addiction disorder, hyperactive gastric motility, ophthalmic conditions, for inducing or maintaining bladder control, and urinary incontinence.

In a preferred embodiment the compounds of the invention are considered useful for treatment, prevention or alleviation of a disease, disorder or adverse condition of the CNS. In a more specific embodiment, the disease, disorder or condition is an affective disorder, a neuro-physiological disorder, anxiety, depression, a bipolar disorder, mania, a sleep disorder, addiction, an eating disorder, a phobia, Parkinson's disease, a mood disorder, a psychotic disorder, a compulsive behaviour, mania, psychosis or schizophrenia.

In a more preferred embodiment the disease, disorder or condition contemplated according to the invention is anxiety.

In another preferred embodiment the compounds of the invention are considered useful for treatment, prevention or alleviation of a CNS damage caused by trauma or by a spinal cord damage, stroke, a neurodegenerative illness or disease, dementia, Alzheimer's disease, a motor neuron disease, a Parkinson-like motor disorder, multiple sclerosis, amyelotrophic lateral sclerosis (ALS), HIV dementia, Huntington's disease, Pick's disease, torsades de pointes, tremor, muscle spasms, myasthenia gravis, convulsions, ataxia, myokymia, seizures, epilepsy or spasticity.

In a third preferred embodiment the compounds of the invention are considered useful for treatment, prevention or alleviation of pain, including acute and chronic pain, neuropathic pain, central pain, or pain related to diabetic neuropathy, to postherpetic neuralgia, to peripheral nerve injury or drug addiction, migraine and migraine-related disorders and to tension-type headache. In a more specific embodiment the pain is somatic pain, incl. visceral pain or cutaneous pain, or pain caused by inflammation or by infection. In another specific embodiment the pain is neuropathic, e.g. caused by injury to the central or peripheral nervous system, e.g. due to tissue trauma, infection, diabetes, an autoimmune disease, arthritis or neuralgia.

In a fourth preferred embodiment the compounds of the invention are considered useful for treatment, prevention or alleviation of a learning and cognitive disorder, memory dysfunction, memory impairment, age-associated memory loss or Down's syndrome.

In a fifth preferred embodiment the compounds of the invention are considered useful for treatment, prevention or alleviation of a disease, disorder or condition associated with the heart or skeletal muscle, heart failure, cardiomyopathia, cardiac arrhythmia, cardiac ischaemia or long QT syndrome.

In a sixth preferred embodiment the compounds of the invention are considered useful for treatment, prevention or alleviation of an inflammatory disease or condition, inflammatory bowel disease, Crohn's disease, ulcerative colitis or Creutzfeld-Jacobs disease.

In a seventh preferred embodiment the compounds of the invention are considered useful for treatment, prevention or alleviation of asthma, an obstructive or inflammatory airway disease, an airway hyper reactivity, a pneumoconiosis such as aluminosis, anthracosis, asbestosis, chalicosis, ptilosis, siderosis, silicosis, tabacosis and byssinosis, a chronic obstructive pulmonary disease (COPD), excerbation of airways hyper reactivity or cystic fibrosis.

In an eight preferred embodiment the compounds of the invention are considered useful for treatment, prevention or alleviation of progressive hearing loss or tinnitus, an ophthalmic disorder, a drug-dependence or drug-addiction disorder, hyperactive gastric motility or urinary incontinence.

In a more preferred embodiment the compounds of the invention are considered useful for treatment, prevention or alleviation of pain, neurodegenerative disorders, migraine, bipolar disorders, mania, epilepsy, convulsions, seizures and seizure disorders, anxiety, depression, functional bowel disorders and multiple sclerosis.

In an even more preferred embodiment the compounds of the invention are considered useful for treatment, prevention or alleviation of pain, including mild, moderate or even severe pain of acute, chronic or recurrent character, as well as neuropathic pain and pain caused by migraine, postoperative pain, phantom limb pain, neuropathic pain, chronic headache, tension type headache, central pain, pain related to diabetic neuropathy, to post therapeutic neuralgia, or to peripheral nerve injury.

In a most preferred embodiment the compounds of the invention are considered useful for treatment, prevention or alleviation of pain, chronic pain, neuropathic pain, epilepsy or anxiety.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the sub-micromolar and micromolar range, i.e. of from below 1 to about 100 μM.

### Pharmaceutical Compositions

Viewed from one aspect the invention relates to the use of a quinazolinone derivative of the invention, or a pharmaceutically-acceptable addition salt thereof, for the manufacture of a pharmaceutical composition for the treatment, prevention or alleviation of a disease or a disorder or a condition of a mammal, including a human, which disease, disorder or condition is responsive to modulation of Kᵥ7 channels.

Viewed from another aspect, the invention provides pharmaceutical compositions comprising a therapeutically-effective amount of a quinazolinone derivative of the invention, or a pharmaceutically-acceptable addition salt thereof, together with at least one pharmaceutically-acceptable carrier or diluent, for the treatment, prevention or alleviation of a disease or a disorder or a condition that is responsive to modulation of Kᵥ7 channels.

While a quinazolinone derivative for use according to the invention may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising a quinazolinone derivative of the invention, together with one or more pharmaceutically acceptable carriers therefore, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route which suite the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition may be prepared by the skilled person using standard and conventional techniques appropriate for the desired formulation. When desired, compositions adapted to give sustained release of the active ingredient may be employed.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration,

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to activation of Kᵥ7 channels, and which method comprises administering to such a living animal body, including a human, in need thereof an effective amount of a quinazolinone derivative of the invention.

The preferred medical indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Example 1

### Preparative Example

The compounds of the invention may be synthesised as outlined in general terms in Scheme 1 and Scheme 2, and described in more detail below.

Both cis and trans isomers of arylcyclopropane carboxylic acids are known and are easily distinguishable by their NMR spectre based on the coupling constants of the vicinal protons. Furthermore cis and trans isomers are easily separated by traditional column chromatography. Generally, Method A describes a method by which the major product formed is the trans arylcyclopropane. Conversely, Method B describes a modified procedure whereby the major products formed are cis isomers. Method C describes a method by which trans isomers are preferred.

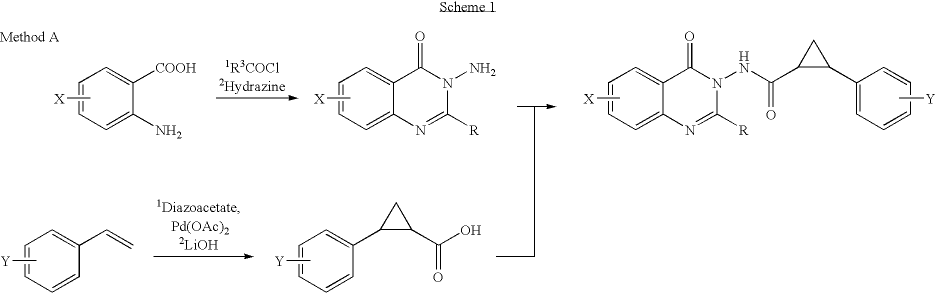

2-(4-Chloro-phenyl)-cyclopropanecarboxylic acid (Intermediate Compound)

4-Chlorostyrene (5 mL; 41.7 mmol) was dissolved in dichloromethane and the solution was purged thoroughly with argon before Pd(OAc)₂ (0.78 g; 3.5 mmol) was added. Finally ethyl diazoacetate (3.6 mL; 34.7 mmol) was slowly added in a drop-wise manner to control nitrogen evolution. After full addition the reaction mixture was stirred for 16 hours at room temperature. The reaction mixture was filtered over silica using 10% EtOAc in heptane and the filtrate was concentrated. The crude oil was redissolved in THF/water 1:1 and lithium hydroxide monohydrate (7.3 g; 174 mmol) was added. The reaction mixture was stirred at room temperature for 16 hours. EtOAc was added and the layers were separated. The water layer was acidified using 4N HCl and extracted with EtOAc. The organic layer was dried on Na₂SO₄ and concentrated to afford 1.3 g (16%) of the title compound as a slightly brown solid.

(Trans)-2-(4-Fluoro-phenyl )-cyclopropanecarboxylic acid (Intermediate Compound)

Similar procedure as for 2-(4-Chloro-phenyl)-cyclopropanecarboxylic acid using 4-fluorostyrene.

3-Amino-2-isopropyl-3H-quinazolin-4-one (Intermediate Compound)

To a solution of 2-aminobenzoic acid (32.7 g; 0.24 mmol) in dry THF (300 mL) was added pyridine (116 mL; 1.4 mol) after which isobutyrylchloride (100 mL; 0.95 mol) over 45 minutes was added in a drop-wise manner. The reaction mixture was refluxed over night after which the reaction mixture was placed on an ice-water bath and hydrazine hydrate (139 mL; 2.86 mol) was added. The reaction mixture was then allowed to reach room temperature and stirred for 4 hours. The reaction mixture evaporated to dryness and the remanesense was added a diethyl ether-benzine (bp=80-100° C.) mixture under stirring by which a white solid precipitates. The white crystals was isolated by filtration and washed with benzine (bp=80-100° C.) to give after drying 25.25 g (52%) pure product.

(Cis)-2-(4-Chloro-phenyl)-cyclopropanecarboxylic acid (2-isopropyl-4-Oxo-4H-Quinazolin-3-yl)-amide (Compound A1)

2-(4-Chloro-phenyl)-cyclopropanecarboxylic acid (0.7 g; 3.6 mmol) was dissolved in dichloromethane and oxalyl chloride (428 μL; 5.0 mmol) was added followed by a drop of DMF (cat.) and gas evolution was observed. The reaction mixture was stirred at room temperature for 1 hour. Pyridine (290 μL; 3.6 mmol) was added followed by 3-Amino-2-isopropyl-3H-quinazolin-4-one (0.72 g; 3.6 mmol) and more pyridine (580 μL; 7.1 mmol) and stirring was continued for 16 hours at room temperature. The reaction mixture was washed with 1N HCl, 1N NaOH and brine. Concentration of the organic layer followed by column chromatography on silica gel with a gradient of 10%-40% EtOAc in heptane afforded 0.38 g (28%) of the title compound as a white solid. Yield 18% LC-ESI-HRMS of [M+H]⁺ shows 382.1305 Da. Calc. 382.13223 Da, dev. −4.5 ppm.

(Trans)-2-(4-Fluoro-Phenyl )-Cyclopropanecarboxylic Acid (2-Isopropyl-4-Oxo-4H-Quinazolin-3-yl)-Amide (Compound A2)

Similar procedure as for 2-(4-chloro-phenyl)-cyclopropanecarboxylic acid (2-isopropyl-4-oxo-4H-quinazolin-3-yl)-amide using (trans)-2-(4-fluoro-phenyl)-cyclopropane-carboxylic acid. Yield 4% LC-ESI-HRMS of [M+H]⁺ shows 366.1602 Da. Calc. 366.16178 Da, dev. −4.3 ppm.

(Trans)-2-(4-Chloro-phenyl)-cyclopropanecarboxylic acid (2-isopropyl-4-oxo-4H-quinazolin-3-yl)-amide (Compound A3)

Similar procedure as for (trans)-2-(4-chloro-phenyl)-cyclopropanecarboxylic acid (2-isopropyl-4-oxo-4H-quinazolin-3-yl)-amide using (trans)-2-(4-chloro-phenyl)-cyclopropanecarboxylic acid. Yield 28% LC-ESI-HRMS of [M+H]+ shows 382.1308 Da. Calc. 382.13223 Da, dev. −3.7 ppm.

(Trans)-2-Phenyl-cyclopropanecarboxylic acid (2-isopropyl-4-oxo-4H-quinazolin-3-yl)-amide (Compound A4)

From commercially available (trans)-2-phenyl-1-cyclopropanecarboxylic acid and using the amide bond formation described in Method A. Yield 39% LC-ESI-HRMS of [M+H]+ shows 348.1706 Da. Calc. 348.171202 Da, dev. −1.7 ppm.

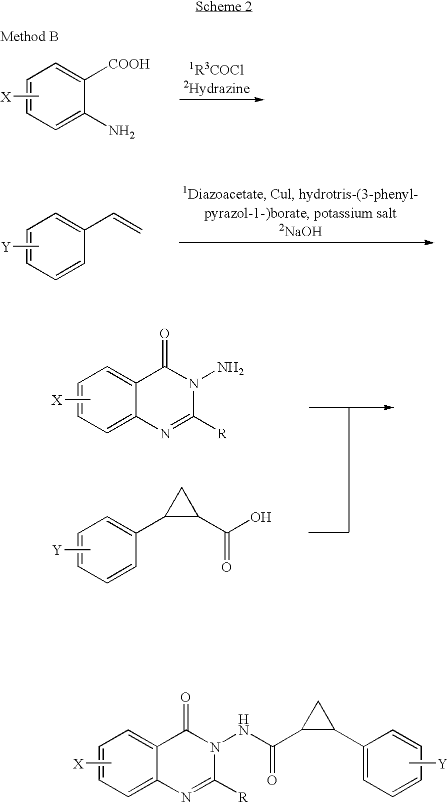

(Cis)-2-(4-Fluoro-phenyl)-cyclopropanecarboxylic acid (Intermediate Compound)

To a suspension of Cul (20 mg; 0.11 mmol) and hydrotris-(3-phenylpyrazol-1-yl)borate,potassium-salt (50 mg; 0.11 mmol) in dry DCM (30 mL) was thoroughly deoxygenated by a evacuation/nitrogen back fill procedure (3 times) after which the suspension was stirred at room temperature for 2 hours. The neat 4-fluorostyrene was added via syringe followed by addition of ethyldiazoacetate and the reaction was stirred overnight at room temperature. The reaction was subsequently quenched with 1M HCl (30 mL) and the organic phase was dried (MgSO₄) and evaporated. The crude product was dissolved in methanol (15 mL) and 4M NaOH was added (15 mL) after which it was refluxed for 2 hours. After cooling, the reaction mixture was diluted with water (30 mL) and extracted with ether (20 mL) to remove styrene. The aqueous phase was made acidic with 4M (HCl) (20 mL) and extracted with DCM (2×20 mL). The combined organic phases were dried (MgSO₄) and evaporated →0.265 g˜59% as a 4:1 mixture of cis and trans.

(Cis)-2-(4-Fluoro-phenyl)-cyclopropanecarboxylic acid (2-isopropyl-4-oxo-4H-quinazolin-3-yl)-amide (Compound B1)

Similar procedure as for (trans)-2-(4-chloro-phenyl)-cyclopropanecarboxylic acid (2-isopropyl-4-oxo-4H-quinazolin-3-yl)-amide using (cis)-2-(4-fluoro-phenyl)-cyclopropanecarboxylic acid. Yield 44% LC-ESI-HRMS of [M+H]+ shows 366.1625 Da. Calc. 366.16178 Da, dev. 2 ppm.

(Cis)-2-P-Tolyl-cyclopropanecarboxylic acid (2-isopropyl-4-oxo-4H-quinazolin-3-yl)-amide (Compound B2)

Similar procedure as for (trans)-2-(4-chloro-phenyl)-cyclopropanecarboxylic acid (2-isopropyl-4-oxo-4H-quinazolin-3-yl)-amide using (cis)-2-p-tolyl-cyclopropanecarboxylic acid. Yield 52%. LC-ESI-HRMS of [M+H]+ shows 362.1884 Da. Calc. 362.186852 Da, dev. 4.3 ppm.

(Cis)-2-(3-Fluoro-phenyl )-cyclopropanecarboxylic acid (2-isopropyl-4-oxo-4H-quinazolin-3-yl)-amide (Compound B3)

Similar procedure as for (trans)-2-(4-chloro-phenyl)-cyclopropanecarboxylic acid (2-isopropyl-4-oxo-4H-quinazolin-3-yl)-amide using (cis)-2-(3-fluoro-phenyl)-cyclopropanecarboxylic acid. Yield 34%. LC-ESI-HRMS of [M+H]+ shows 366.1609 Da. Calc. 366.16178 Da, dev. −2.4 ppm.

(Trans)-2-p-tolyl-cyclopropanecarboxylic acid (2-isopropyl-4-oxo-4H-quinazolin-3-yl)-amide (Compound B4)

From the synthesis of (cis)-2-(3-fluoro-phenyl)-cyclopropanecarboxylic acid, some (trans)-2-(3-fluoro-phenyl)-cyclopropanecarboxylic acid was isolated which was used to prepare the title compound by a similar procedure as for (trans)-2-(4-chlorophenyl)-cyclopropanecarboxylic acid (2-isopropyl-4-oxo-4H-quinazolin-3-yl)-amide using (cis)-2-(3-fluoro-phenyl)-cyclopropanecarboxylic acid. Yield 14% LC-ESI-HRMS of [M+H]+ shows 362.1884 Da. Calc. 362.186852 Da, dev. 4.3 ppm.

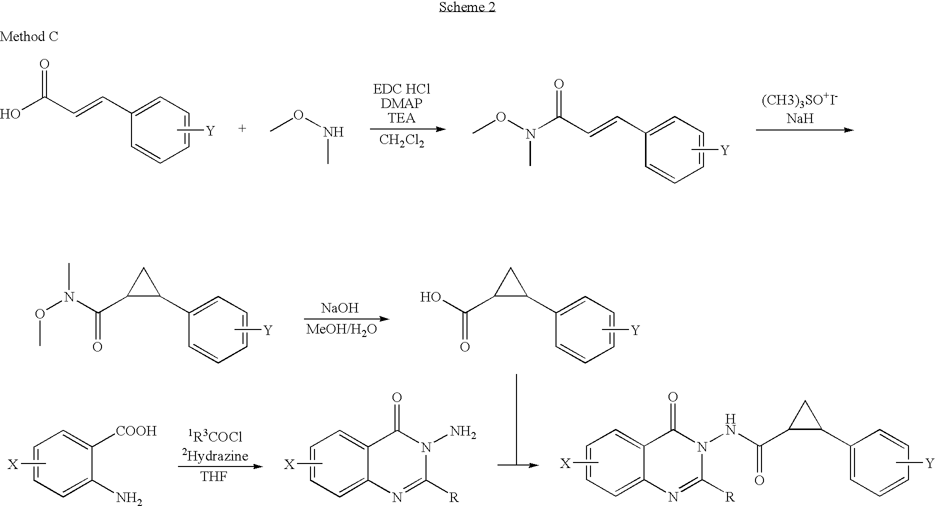

3-(3-Fluoro-4-methyl-phenyl)-N-methoxy-N-methyl-acrylamide (Intermediate Compound)

To a solution of 3-fluoromethylcinnamic acid (4.87 g; 27 mmol) in methylenechloride (60 mL) was added 4-dimethylaminopyridine (3.3 g; 27 mmol), 1-(3-Dimethylaminopropyl)-3-ethylcarbodiimide,hydrochloride (5.7 g; 29.7 mmol) triethylamine (7.5 mL; 54 mmol) and eventually N,O-dimethylhydroxylamine hydrochloride (2.89 g; 47.3 mmol) and the mixture was stirred for 2 days. The reaction mixture was washed with H₂O (200 mL), 1 M HCl (100 mL) and sat. NaHCO₃ (100 mL) after which it was dried (MgSO₄), filtered and evaporated to dryness yielding 5,92 g (98%) of a brownish liquid which was taken to the next step without further purification.

2-(3-Fluoro-4-methyl-phenyl)-cyclopropanecarboxylic acid methoxy-methyl-amide (Intermediate Compound)

To a solution of trimethylsulfoxonium iodide (11.67 g; 53 mmol) in DMF (50 mL) at 0° C. was added sodium hydride (60% dispersion; 2.12 g; 53 mmol). The slurry was allowed to reach room temperature and stirred for another 45 minutes after which the mixture was re-cooled to 0° C., at which temperature a solution of 3-(3-fluoro-4-methyl-phenyl)-N-methoxy-N-methyl-acrylamide (5.92 g; 26.5 mmol) in DMF (5 mL) was added. The resulting mixture was allowed to reach room temperature at which temperature it was stirred for 2 hours. The reaction mixture was poured into water (100 mL) and added CH₂Cl₂ (100 mL). The organic phase was washed and washed with brine (2×100 mL), dried (MgSO₄), filtered, dried and evaporated to give 16 g. The crude reaction mixture was washed with water (4×50 mL), dried (MgSO₄), filtered, dried and evaporated to give 5.4 g which was further purified by Combiflash Sq16 (120 g kiselgel column; eluent 100% Benzine (bp=80-100° C.) to Benzine (bp=80-100° C.)/EtOAc=1:1 over 20 minutes) to give 3.7 g (59%) pure product as a clear liquid.

2-(3-Fluoro-4-methyl-phenyl)-cyclopropanecarboxylic acid (Intermediate Compound)

To a solution of 2-(3-fluoro-4-methyl-phenyl)-cyclopropanecarboxylic acid methoxy-methyl-amide (3.7 g; 15.6 mmol) in MeOH (40 mL) and water (20 mL) was added sodium hydroxide (1.5 g; 37.5 mmol) after which the mixture was refluxed for 3 hours. The reaction mixture was concentrated in vacuo and made acidic using 4M HCl (aq.). The precipitated material was extracted using CH₂Cl₂ (30 mL) and the aqueous phase extracted once more with CH₂Cl₂ (30 mL). The combined organic fractions were dried (MgSO₄), filtered and evaporated to dryness giving a quantitative yield of pure product (3.02 g; 100%).

(Trans)-2-(3-Fluoro-4-methyl-phenyl)-cyclopropanecarboxylic acid (2-isopropyl-4-oxo-4H-quinazolin-3-Yl)-amide (Compound C1)

To a solution of 2-(3-fluoro-4-methyl-phenyl)-cyclopropanecarboxylic acid (0.24 g; 1.23 mmol) CH₂Cl₂ (10 mL) was added oxalylchloride (0.6 mL; 6.9 mmol) and two drops of dry dimethylformamide and the mixture was left with stirring for 60 minutes at room temperature. Pyridine (80 μL; 1.02 mmol) was added, then 3-amino-2-isopropyl-3H-quinazolin-4-one (0.21 g; 1.03 mmol) and more pyridine (160 μL; 2.02 mmol) and the mixture was stirred over night after which the reaction was quenched using 1M HCl (aq.; 10 mL). The organic phase was dried (MgSO₄), filtered and evaporated onto Celite. The crude product was purified by CombiFlash SQ16 [4g kiselgel column; benzine (bp=80-100° C.)/EtOAc=9:1 going to 100% EtOAc over 12minutes and the on a 12 g kiselgel column using bp=80-100° C.)/EtOAc=9:1 going to 50% EtOAc over 16 minutes] to give after evaporation 164 mg (42%) pure product as a white solid.

### Example 2

### Biological Activity

In a standard patch-clamp set-up, e.g. as outlined in International Patent Publication WO 2004/080377, using CHO K1 cell lines stably expressing the human Kᵥ7₂₊₃ channels, the compounds of the invention were found to be activators of the channels at various concentrations at various degrees. For example, at a concentration of 3 μM, Compound A4 induces an increase in current amplitude in the order 190%, which is an indication of its potent Kᵥ7₂₊₃ activating activity.

1. A quinazolinone derivative of Formula I 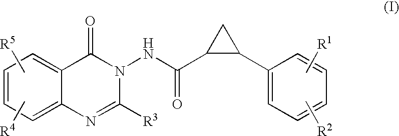
any of its stereoisomers, or any mixture of its stereoisomers, or a pharmaceutically-acceptable addition salt thereof, or an N-oxide thereof, wherein
R¹ and R², independently of each other, represent hydrogen, alkyl, cycloalkyl, halo, haloalkyl, hydroxy, alkoxy, baloalkoxy, amino, alkyl-carbonyl-amino, alkyl-sulfonyl, cyano or nitro;
R³ represents alkyl, cycloalkyl or alkoxy; and
R⁴ and R⁵, independently of each other, represent hydrogen, alkyl, cycloalkyl, halo, haloalkyl, hydroxy, alkoxy, haloalkoxy, amino, alkyl-carbonyl-amino, nitro or cyano.

2. The quinazolinone derivative of claim 1, or a pharmaceutically-acceptable addition salt thereof, or an N-oxide thereof, wherein
R¹ and R², independently of each other, represent hydrogen, alkyl, cycloalkyl, halo, haloalkyl, hydroxy, alkoxy, haloalkoxy, amino, alkyl-carbonyl-amino, alkyl-sulfonyl, cyano or nitro.

3. The quinazolinone derivative of claim 1 or a pharmaceutically-acceptable addition salt thereof, or an N-oxide thereof, wherein
R³ represents alkyl, cycloalkyl or alkoxy.

4. The quinazolinone derivative of claim 1, or a pharmaceutically-acceptable addition salt thereof, or an N-oxide thereof, wherein
R⁴ and R⁵, independently of each other, represent hydrogen, alkyd, cycloalkyl, halo, haloalkyl, hydroxy, alkoxy, haloalkoxy, amino, alkyl-carbonyl-amino, nitro or cyano.

5. The quinazolinone derivative of claim 1, which is
2-(4-Chloro-phenyl)-cyclopropanecarboxylic acid (2-isopropyl-4-oxo-4H-quinazolin-3-yl)-amide;
2-(4-Fluoro-phenyl)-cyclopropanecarboxylic acid (2-isopropyl-4-oxo-4H-quinazolin-3-yl)-amide;
2-(3-Fluoro-4-methyl-phenyl)-cyclopropanecarboxylic acid (2-isopropyl-4-oxo-4H-quinazolin-3-yl)- amide;
2-Phenyl-cyclopropanecarboxylic acid (2-isopropyl-4-oxo-4H-quinazolin-3-yl)-amide;
2-(4-Chloro-phenyl)-cyclopropanecarboxylic acid (2-isopropyl-4-oxo-4H-quinazolin-3-yl)-amide;
2-(⁴-Fluoro-phenyl)-cyclopropanecarboxylic acid (2-isopropyl-4-oxo-4H-quinazolin-3-yl)-amide;
2-p-Tolyl-cyclopropanecarboxylic acid (2-isopropyl-4-oxo-4H-quinazolin-3-yl)-amide;
2-(3-Fluoro-phenyl)-cyclopropanecarboxylic acid (2-isopropyl-4-oxo-4H-quinazolin-3-yl)-amide; or
2-p-Tolyl-cyclopropanecarboxylic acid (2-isopropyl-4-oxo-4H-quinazolin-3-yl)-amide;
or a pharmaceutically-acceptable addition salt thereof,

6. A pharmaceutical composition comprising a therapeutically effective amount of the quinazolinone derivative of claim 1, or a pharmaceutically-acceptable addition salt thereof; or a prodrug thereof, together with one or more adjuvants, excipients, carriers and/or diluents.

7. A method of treatment, prevention or alleviation of a disease or a disorder or a condition of a living animal body, including a human which disorders disease or condition is responsive to activation of Kᵥ7 channels, which disorder, disease or condition is reponsive to activation of Kᵥ7 channels, which method comprises the step of administering to such a living animal body in need thereof, a therapeutically effective amount of the quinazolinone derivative of claim 1, a pharmaceutically-acceptable addition salt thereof, or a prodrug thereof.

8. The method according to claim 7, wherein the disease, disorder or condition is an affective disorder, neuro-physiological disorder, anxiety, depression, a bipolar disorder, mania, a sleep disorder, addiction, an eating disorder, a phobia, Parkinson's disease, a mood disorder, a psychotic disorder, a compulsive behaviour, mania, psychosis, schizophrenia, dementia, Alzheimer's disease, epilepsy, convulsions, seizures, seizure disorders, tremor, muscle spasms, myasthenia gravis, a motor neuron disease, motion and motor disorders, a Parkinson-like motor disorder, multiple sclerosis, amyelotrophic lateral sclerosis (ALS), HIV dementia, Huntington's disease, Pick's disease, torsades de pointes, functional bowel disorders, neurodegenerative disorders, CNS damage caused by trauma, stroke or neurodegenerative illness or diseases, ataxia, myokymia, spasticity, learning and cognitive disorders, memory dysfunction, memory impairment, age-associated memory loss, Down's syndrome, pain, acute or chronic pain, mild pain, moderate or severe pain, neuropathic pain, central pain, pain related to diabetic neuropathy, to postheipetic neuralgia, to peripheral neive injury or to drug addiction, somatic pain, visceral pain or cutaneous pain pain caused by inflammation or by infection, postoperative pain, phantom limb pain, chronic headache, migraine, migraine-related disorders, tension-type headache, heart failure, cardiac disorders, cardiomyopathia, cardiac arrhythmia, cardiac ischaemia, long QT syndrome, inflammatory diseases or conditions, inflammatory bowel disease, Crohn's disease, ulcerative colitis, Creutzfeld-Jacobs disease, an obstructive or inflammatory airway disease, asthma, an airway hyper reactivity, pneumlocoliosis, aluminosis, anthracosis, asbestosis, chalicosis, ptilosis, siderosis, silicosis, tabacosis, byssinosis, chronic obstructive pulmonary disease (COPD), excerbation of airways hyper reactivity, cystic fibrosis, progressive hearing loss, tinnitus, a drug-dependence or drug-addiction disorder, hyperactive gastric motility, ophthalmic conditions, for inducing or maintaining bladder control, or urinary incontinence.

9. (canceled)
